SEQUENCE LISTING (2 sequences)

SEQ ID NO: 1 - DNA, 2856 bp, Homo sapiens
  polyA_signal (2801)..(2806): MTA1 gene
gcggccctcccgtccctgcgcggcctcggcggcctcggcggcggcggcggcggcggcggcggcagcagcgcggcccctttaaacgcctgcggcgccccccgcccccgccatcgcgcctccattttcccggccgcccgcgccgagcgccgcgcccgccccgggcccctccgccgccgccggcccggacatggccgccaacatgtacagggtcggagactacgtctactttgagaactcctccagcaacccatacctgatccggagaatcgaggagctcaacaagacggccaatgggaacgtggaggccaaagtggtgtgcttctaccggaggcgggacatctccagcaccctcatcgccctggccgacaagcacgcaaccctgtcagtctgctataaggccggaccgggggcggacaacggcgaggaaggggaaatagaagaggaaatggagaatccggaaatggtggacctgcccgagaaactaaagcaccagctgcggcatcgggagctgttcctctcccggcagctggagtctctgcccgccacgcacatcaggggcaagtgcagcgtcaccctgctcaacgagaccgagtcgctcaagtcctacctggagcgggaggatttcttcttctattctctagtctacgacccacagcagaagaccctgctggcagataaaggagagattcgagtaggaaaccggtaccaggcagacatcaccgacttgttaaaagaaggcgaggaggatggccgagaccagtccaggttggagacccaggtgtgggaggcgcacaacccactcacagacaagcagatcgaccagttcctggtggtggcccgctctgtgggcaccttcgcacgggccctggactgcagcagctccgtccgacagcccagcctgcacatgagcgccgcagctgcctcccgagacatcaccctgttccacgccatggatactctccacaagaacatctacgacatctccaaggccatctcggcgctggtgccgcagggcgggcccgtgctctgcagggacgagatggaggagtggtctgcatcagaggccaaccttttcgaggaagccctggaaaaatatgggaaggatttcacggacattcagcaagattttctcccgtggaagtcgctgaccagcatcattgagtactactacatgtggaagaccaccgacagatacgtgcagcagaaacgcttgaaagcagctgaagctgagagcaagttaaagcaagtttatattcccaactataacaagccaaatccgaaccaaatcagcgtcaacaacgtcaaggccggtgtggtgaacggcacgggggcgccgggccagagccctggggctggccgggcctgcgagagctgttacaccacacagtcttaccagtggtattcttggggtccccctaacatgcagtgtcgtctctgcgcatcttgttggacatattggaagaaatatggtggcttgaaaatgccaacccggttagatggagagaggccaggaccaaaccgcagtaacatgagtccccacggcctcccagcccggagcagcgggagccccaagtttgccatgaagaccaggcaggctttctatctgcacacgacgaagctgacgcggatcgcccggcgcctgtgccgtgagatcctgcgcccgtggcacgctgcgcggcacccctacctgcccatcaacagcgcggccatcaaggccgagtgcacggcgcggctgcccgaagcctcccagagcccgctggtgctgaagcaggcggtacgcaagccgctggaagccgtgcttcggtatcttgagacccacccccgcccccccaagcctgaccccgtgaaaagcgtgtccagcgtgctcagcagcctgacgcccgccaaggtggcccccgtcatcaacaacggctcccccaccatcctgggcaagcgcagctacgagcagcacaacggggtggacggcaacatgaagaagcgcctcttgatgcccagtaggggtctggcaaaccacggacaggccaggcacatgggaccaagccggaacctcctgctcaacgggaagtcctaccccaccaaagtgcgcctgatccgggggggctccctgcccccagtcaagcggcggcggatgaactggatcgacgccccggatgacgtgttctacatggccacagaggagaccaggaagatccgcaagctgctctcatcctcggaaaccaagcgtgctgcccgccggccctacaagcccatcgccctgcgccagagccaggccctgccgccgcggccaccgccacctgcgcccgtcaacgacgagcccatcgtcatcgaggactaggggccgcccccacctgcggccgccccccgcccctcgcccgcccacacggccccttcccagccagcccgccgcccgcccctcagtttggtagtgccccacctcccgccctcacctgcagagaaacgcgctccttggcggacactgggggaggagaggaagaagcgcggctaacttattccgagaatgccgaggagttgtcgtttttagctttgtgtttactttttggctggagcggagatgaggggccaccccgtgcccctgtgctgcggggccttttgcccggaggccgggccctaaggttttgttgtgttctgttgaaggtgccattttaaattttatttttattactttttttgtagatgaacttgagctctgtaacttacacctggaatgttaggatcgtgcggccgcggccggccgagctgcctggcggggttggcccttgtcttttcaagtaattttcatattaaacaaaaacaaagaaaaaaaatcttataaaaaggaaaaaaaaaaaaaaaaaa

SEQ ID NO: 2 - protein, 715 aa, Homo sapiens
  PEPTIDE (1)..(715): MTA1 protein
MAANMYRVGDYVYFENSSSNPYLIRRIEELNKTANGNVEAKVVCFYRRRDISSTLIALADKHATLSVCYKAGPGADNGEEGEIEEEMENPEMVDLPEKLKHQLRHRELFLSRQLESLPATHIRGKCSVTLLNETESLKSYLEREDFFFYSLVYDPQQKTLLADKGEIRVGNRYQADITDLLKEGEEDGRDQSRLETQVWEAHNPLTDKQIDQFLVVARSVGTFARALDCSSSVRQPSLHMSAAAASRDITLFHAMDTLHKNIYDISKAISALVPQGGPVLCRDEMEEWSASEANLFEEALEKYGKDFTDIQQDFLPWKSLTSIIEYYYMWKTTDRYVQQKRLKAAEAESKLKQVYIPNYNKPNPNQISVNNVKAGVVNGTGAPGQSPGAGRACESCYTTQSYQWYSWGPPNMQCRLCASCWTYWKKYGGLKMPTRLDGERPGPNRSNMSPHGLPARSSGSPKFAMKTRQAFYLHTTKLTRIARRLCREILRPWHAARHPYLPINSAAIKAECTARLPEASQSPLVLKQAVRKPLEAVLRYLETHPRPPKPDPVKSVSSVLSSLTPAKVAPVINNGSPTILGKRSYEQHNGVDGNMKKRLLMPSRGLANHGQARHMGPSRNLLLNGKSYPTKVRLIRGGSLPPVKRRRMNWIDAPDDVFYMATEETRKIRKLLSSSETKRAARRPYKPIALRQSQALPPRPPPPAPVNDEPIVIED

(19) United States
(12) Patent Application Publication
(10) Pub. No.: US 2011/0171661 A1
(43) Pub. Date: Jul. 14, 2011

(54) METHOD FOR DIAGNOSIS OF POST-OPERATIVE RECURRENCE IN PATIENTS WITH HEPATOCELLULAR CARCINOMA

(76) Inventors: Young Hwa Chung, Seoul (KR); Eun Sil Yu, Seoul (KR); Soo Hyung Ryu, Seoul (KR); Kyu Won Kim, Seoul (KR); Jeong A Kim, Seoul (KR)

(21) Appl. No.: 13/057,029

(22) PCT Filed: Aug. 26, 2008
(86) PCT No.: PCT/KR2008/004996

§ 371 Date: Feb. 1, 2011

(30) Foreign Application Priority Data
Aug. 18, 2008 (KR) 10-2008-0080441

(51) Int. Cl. G01N 33/53

(52) U.S. Cl. 435/7.1

(57) ABSTRACT

Provided are a method of diagnosing the recurrence or possibility of recurrence of hepa [symbol] tocellular carcinoma after hepatocellular carcinoma surgery and a method of screening a drug for preventing the recurrence of hepatocellular carcinoma after hepatocellular carcinoma surgery and treating hepatocellular carcinoma. Specifically, the recurrence or possibility of recurrence of hepatocellular carcinoma after hepatocellular carcinoma surgery is diagnosed and a drug for preventing the recurrence of hepatocellular carcinoma after surgery and treating hepatocellular carcinoma is screened, by measuring expression levels of a metastatic tumor antigen 1 or metastasis associated 1 (MTA1) protein in a biological sample taken from a patient in which hep [symbol] atocellular carcinoma recurs or is likely to recur after hepatocellular carcinoma surgery and comparing the measured expression levels of the MTA1 protein with those of a normal control group.

### TECHNICAL FIELD

The present invention relates to a method of diagnosing the recurrence or possibility of recurrence of hepatocellular carcinoma after hepatocellular carcinoma surgery and a method of screening a drug for preventing the recurrence of hepatocellular carcinoma after hepatocellular carcinoma surgery and treating hepatocellular carcinoma.

### BACKGROUND ART

Hepatocellular carcinoma (HCC) is the 5 ^{th} most common cancer in the world. Although surgery is one of the best treatment modalities for HCC, fewer than 10% to 20% are candidates for surgery because of unresectable size and number of tumors, poor liver functions, and multiple intrahepatic or distant metastasis.

Even in HCC patients who are good candidates for surgery, frequent recurrence after surgery is a major limitation to long-term survival.

Various metastasis-associated proteins of cancers have been studied and isolated. Among them, the recently identified metastatic tumor antigen 1 or metastasis associated 1(MTA1) is known to increase the migration and invasion of various tumor cells in vitro.

MTA1 has also been suggested to play a role in angiogenic processes as a stabilizer of hypoxia-inducible factor 1α(HIF1α).

However, few data are available regarding the role of MTA 1 in invasion or recurrence of HCC, and survival of HCC patients.

To solve problems of the conventional technique, the inventors of the present invention have found that as a metastatic tumor antigen 1 or metastasis associated 1(MTA1), which is known to promote angiogenesis in tumors and to be associated with growth and metastasis of tumors, is more expressed, the recurrence of HCC after surgery more frequently occurs and the patient survival rate is lower.

### DISCLOSURE OF INVENTION

### Technical Problem

The present invention provides a method of diagnosing the recurrence or possibility of recurrence of hepatocellular carcinoma (HCC) after HCC surgery, by using expression levels of a metastatic tumor antigen 1 or metastasis associated 1(MTA1) that is a useful prognostic factor through which the recurrence of HCC and poor survival after HCC surgery can be predicted.

The present invention also provides a method of screening a drug for preventing the recurrence of HCC after surgery and treating HCC.

### TECHNICAL SOLUTION

According to an aspect of the present invention, there is provided a method of diagnosing the recurrence or possibility of recurrence of HCC after HCC surgery, the method including: measuring expression levels of a metastatic tumor antigen 1 or metastasis associated 1(MTA1) protein in a biological sample taken from a patient in which HCC recurs or is likely to recur after HCC surgery; and comparing the measured expression levels of the MTA1 protein with those of a normal control group.

The amino acid sequence of the MTA1 protein is set forth in SEQ ID NO: 2 and is available from GenBank (Registration No. NM_{—}004689.3, GI:115527079).

Regarding MTA1 protein expression according to the present invention, expression levels in the biological sample taken from the patient in which HCC recurs or is likely to recur after HCC surgery are determined as positive or negative according to whether expression occurs or does not occurs when compared to the normal control group. Such determination is relatively made compared to the normal control group by physicians who do not know diagnosis terms. To remove background errors that may occur when staining, less than 25% is determined as negative and 25% or more is determined as positive.

According to another aspect of the present invention, there is provided a method of screening a drug for preventing the recurrence of HCC after HCC surgery and treating HCC, the method including: contacting a metastatic tumor antigen 1 or metastasis associated 1(MTA1) protein with candidate drugs (step 1); measuring expression levels of the MTA1 protein that have contacted the candidate drugs (step 2); measuring expression levels of the MTA1 protein in the absence of the candidate drugs (step 3); and comparing the expression levels of step 2 with the expression levels of step 3 to select a candidate drug that reduces expression levels of the MTA1 protein (step 4).

A pharmaceutical composition including the candidate drug that is selected by screening candidate drugs inhibits MTA1 expression. Accordingly, the pharmaceutical composition can be used to prevent the recurrence of HCC after HCC surgery and treat HCC.

### ADVANTAGEOUS EFFECTS

According to the present invention, by analyzing expression levels of MTA1 based on the founding that high expression levels of a metastatic tenor antigen 1 or metastasis associated 1(MTA1) that are known to promote angiogenesis in tumors and to be associated with growth and metastasis of tumors are closely correlated to the recurrence of HCC and survival after HCC surgery, the recurrence or possibility of recurrence of HCC after HCC surgery can be diagnosed and a drug for preventing the recurrence of HCC or treating HCC can be screened and thus, expression levels of MTA 1 can be usefully used in preventing the recurrence of HCC after surgery and treating HCC.

### BRIEF DESCRIPTION OF DRAWINGS

The above and other features and advantages of the present invention will become more apparent by describing in detail exemplary embodiments thereof with reference to the attached drawings in which:

FIG. 1 is an image to identify expression levels of a metastatic tumor antigen 1 or metastasis associated 1(MTA1) that are immunohistochemically stained in hepatocellular carcinoma (HCC) samples;

FIG. 2 is a graph of MTA1 expression levels with respect to a tumor size;

FIG. 3 is a graph of MTA1 expression levels with respect to a tumor type;

FIG. 4 is a graph of MTA1 expression levels with respect to histological differentiation;

FIG. 5 is a graph of MTA1 expression levels with respect to microvascular emboli;

FIG. 6 is a graph of MTA1 expression levels with respect to a cause of HCC;

FIG. 7 is a graph of HCC cumulative recurrence rates with respect to MTA1 expression levels;

FIG. 8 is a graph of cumulative survival rates of HCC patients with respect to MTA1 expression levels;

FIG. 9 is a graph of HCC cumulative recurrence rates with respect to MTA1 expression levels in hepatitis B virus (HBV) patients; and

FIG. 10 is a graph of cumulative survival rates of HCC patients with respect to MTA1 expression levels in HBV patients.

### BEST MODE FOR CARRYING OUT THE INVENTION

The present invention will be described in further detail with reference to the following examples. These examples are for illustrative purposes only and are not intended to limit the scope of the present invention.

### EXAMPLE 1

### Characteristics of Patients

A total of 506 HCC patients who were treated with hepatic resection during the time period of 1998 to 2003 at the Asan Medical Center were tested. The clinical characteristics of the 506 patients are listed on Table 1. The patients were followed-up for a median period of 43 months (in the range of 1-96 months) after hepatectomy.

TABLE 1

| Characteristic | Value |
|---|---|
| Age (years, mean ± SD) | 56 ± 0 |
| Sex (M:F) | 412:94 |
| Disease severity (CH:LC) | 138:368 |
| Child-Pugh class (A/B/C) | 383/73/50 |
| Causes of HCC [hepatitis B virus (HBV)/hepatitis C virus | 397/29/8/72 |
| (HCV)/Both/non-viral hepatitis cause of HCC (NBNC)] | |
| Size of HCC (cm) | 4 (0.7-21) |
| Follow-up after hepatectomy (months) | 43 (1-96) |

The recurrence and survival were determined using medical records at the last follow-up data. In cases in which a patient was lost to follow-up for more than 3 moths, the recurrence and survival were evaluated based on information obtained by visiting places near the patient's residence.

### EXAMPLE 2

### Immunohistochemical Staining of MTA1

### 1. Tissue Microarray Construction

Tissue microarrays were constructed using a previously known method (refer to Oncol Rep. 16: 929-935, 2006). Formalin-fixed, paraffin-embedded tissue samples were arrayed using a tissue-arraying instrument (refer to Beecher Instruments, Slyer Spring, Md.).

Briefly, representative areas of each tumor were selected and marked on the hematoxylin-eosin (H&E)-stained slide, and its corresponding tissue block was sampled. The target region of each donor block was punched with a tissue cylinder of 1 mm diameter, and the sample was transferred to a recipient block. Each sample was arrayed in duplicate to minimize tissue loss.

### 2. Immunohistochemical Staining of MTA1 in Human HCC Tissue Microarray

Immunohistochemical staining for MTA1 was performed using the avidin-biotin-peroxidase complex method with an LSAB kit (DAKO, Carpinteria, Calif.) and 3,3′-diaminobenzidine as a chromogen. Paraffin-embedded tissue-microarray blocks, which included HCC and surrounding non-neoplastic liver tissues, were sectioned at 5 μm intervals.

Slides were deparaffinized with xylene and rehydrated in a series of alcohol. The slides were then incubated in 3% hydrogen peroxide for 10 minutes to block endogenous peroxidase activity. To increase the immunoreactivity, antigen retrieval was performed in citrate buffer (pH 6.0) for 10 minutes in a steam oven. The primary antibody against MTA1 was used at a dilution of 1:200. Subsequently, secondary biotinylated antibody and avidin-biotin complex reagent were applied and the sections were counterstained with Harris hematoxylin.

For a negative control, sections were incubated with Tris-buffered saline containing 2% goat serum and 1% bovine serum albumin instead of primary antibody.

### 3. Evaluation of Immunostaining

The inventors of the present invention examined a characteristic of tumor cells that were positive for MTA1 in the tumor cell nuclei. For each spot, areas of the most intense or predominant staining patterns were recorded. Their staining intensity was usually positively correlated with the proportion of positive tumor cells. That is, the staining values appeared to cluster in a group of cases with less than half of weakly positive tumor cells or in a group of cases with more than 90% of strongly positive tumor cells.

Based on the findings, the inventors of the present invention used the criteria to simplify the classification and analyze the clinical data effectively; (1) 0% (none, −); (2) MTA1 low group (less than 50%, +); and (3) MTA1 high group (more than 50%, ++).

The nuclear staining is diffused and there were no other staining patterns such as membranous, nucleolar, or speckled patterns that can be observed in cases of another nuclear proteins. Two independent observers determined MTA1 expression levels using the arrays, and when the observers obtained different results, they reexamined the specimens with discrepant scores to determine a consensus score.

### 4. Statistical Analysis

To avoid confusion of analysis and focus on the role of MTA1 as a prognostic factor in HCC recurrence and overall survival, the inventors of the present invention performed experiments using the following standards: first, only objective evidences such as imaging studies were used as HCC recurrence for cumulative recurrence rates and the probability of recurrence was not considered as HCC recurrence; second, all deaths of patients with HCC combined with or without progressed liver dysfunction were considered for cumulative survival rates (overall survival). The inventors of the present invention did not include nonhepatic origin deaths in determining cumulative survival rates. Based on the same criteria, multivariate analysis for recurrence and survival was performed using a Cox regression hazard model.

The survival rate analysis was performed using a Kaplan-Meier method, a univariate analysis was performed using a long-rank test, and a multivariate analysis was performed using the Cox regression hazard model.

### 5. Results

1) MTA1 expression frequencies in HCC and surrounding liver tissues

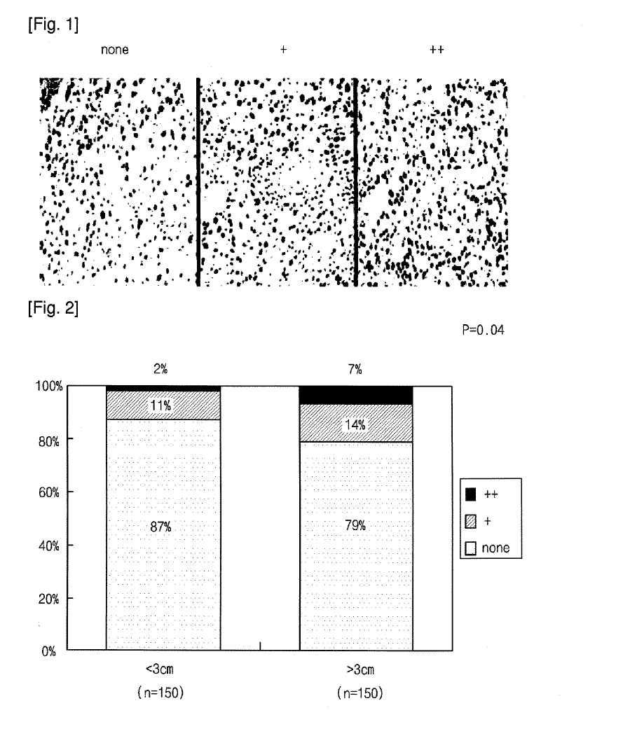

As illustrated in FIG. 1, of the 506 HCC samples, MTA1 was stained in 88 (17%), but none of the surrounding liver tissues were stained. Of positive 88 samples, 62 were + and 26 were ++ (p<0.001).

2) Tumor size and MTA1 expression levels

The level of MTA1 expression was higher in HCC patients with larger tumors. As illustrated in FIG. 2, 150 patients had HCC having a diameter of less than 3 cm, and 333 patients had HCC having a diameter of more than 3 cm. Of the HCC having a diameter of less than 3 cm, MTA1 expression was negative in 87%, + in 11%, and ++ in 2%. Of the HCC having a diameter of more than 3 cm, MTA1 expression was negative in 79%, + in 14%, and ++ in 7% (p=0.04).

3) Tumor type and MTA1 expression levels

The tumor types were analyzed in 434 cases. A nodular type was noted in 263 patients, nodular with perinodal extension in 74 patients, multinodular confluent in 70 patients, pedunculated in 6 patients, and diffuse infiltrative in 21 patients.

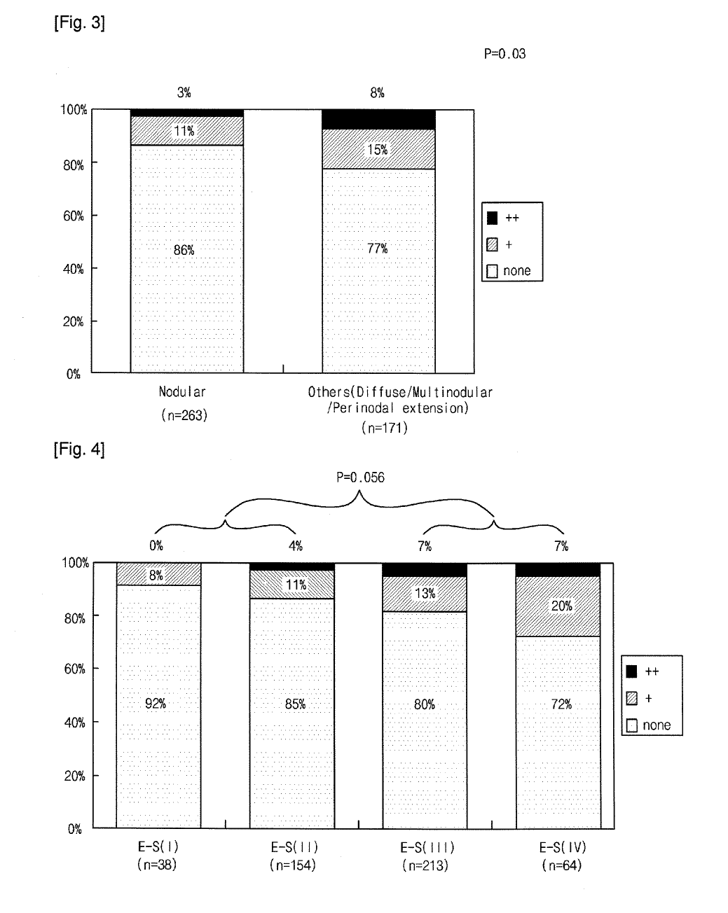

As illustrated in FIG. 3, the level of MTA1 expression was lower in the nodular type of HCC than in the other types of HCC.

4) Histological differentiation and MTA1 expression levels

A histological differentiation analysis was performed on a total of 469 HCC samples. As illustrated in FIG. 4, almonson-Steiner (E-S) grades 1, 2, 3 and 4 were noted in 38, 154, 213 and 64 patients, respectively. MTA1 expression levels in E-S grade 1 were negative in 92%, + in 8%, and ++ in 0%. MTA1 expression levels in E-S grade 2 were negative in 85%, + in 11%, and ++ in 4%. MTA1 expression levels in E-S grade 3 were negative in 80%, + in 13%, and ++ in 7%. MTA1 expression levels in E-S grade 4 were negative in 72%, + in 20%, and ++ in 7%.

It was assumed that increased MTA1 expression levels were associated with worse histological differentiation of HCC.

5) Microvascular emboli and MTA1 expression levels

Microvascular emboli in 452 HCC frozen tissues were examined. As a result, as illustrated in FIG. 5, 102 (22.6%) showed microvascular emboli. MTA1 expression levels in patients with microvascular emboli in frozen tissues were much higher than those of patients without microvascular emboli.

6) The causes of HCC and MTA1 expression levels

Different causes of liver disease were associated with differences in MTA1 expression levels. Of the 484 patients, 380 had hepatitis B virus (HBV), 27 had hepatitis C virus (HCV), and 8 had both HBV and HCV. A total of 69 patients had a non-viral hepatitis cause (NBNC) of HCC.

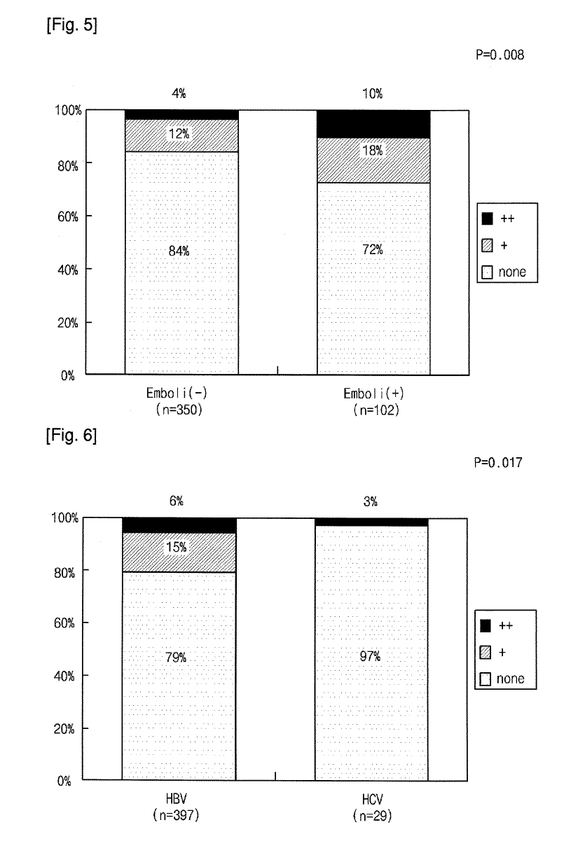

Interestingly, MTA1 was expressed in 80 of 380 patients with HBV-associated HCC (21%), but in only 1 patient (4%) with HCV-associated HCC. That is, as illustrated in FIG. 6, the levels of MTA1 expression in HBV-associated HCC were negative in 79%, + in 15%, and ++ in 6%, and MTA1 expression levels in HCV-associated HCC were negative in 96%, + in 0%, and ++ in 4%.

None of HCCs from the patients with HBV and HCV co-infection had MTA1 expression (0%, 0/8). Among 72 HCC patients with nonviral causes, 69 patients were available for evaluation of MTA1 staining. Of 69 patients, 7% (5/69) had level 1 (+) expression of MTA1 and 3% (2/69) had level 2 (++) expression of MTA1.

7) Correlation of other clinicopathological factors and MTA1 expression levels

As shown in Table 2, there was no association between MTA1 expression levels and age, sex, Child-Pugh class of liver disease, decompensation of liver function, or capsule invasion of HCC. In Table 2, PVT is an abbreviation of portal vein thrombosis.

TABLE 2

| Variables | Negative | + | ++ | P Values |
|---|---|---|---|---|
| Age (years) | 57 (4-88) | 58 (34-76) | 57 (29-71) | NS |
| Male (%) | 81 | 86 | 85 | NS |
| Capsule invasion (%) | 21 | 29 | 22 | NS |
| PVT (%) | 13 | 13 | 12 | NS |
| Cirrhosis (%) | 70 | 64 | 67 | NS |
| Decompensation (%) | 25 | 26 | 16 | NS |

8) Recurrence and survival rates according to MTA1 expression levels

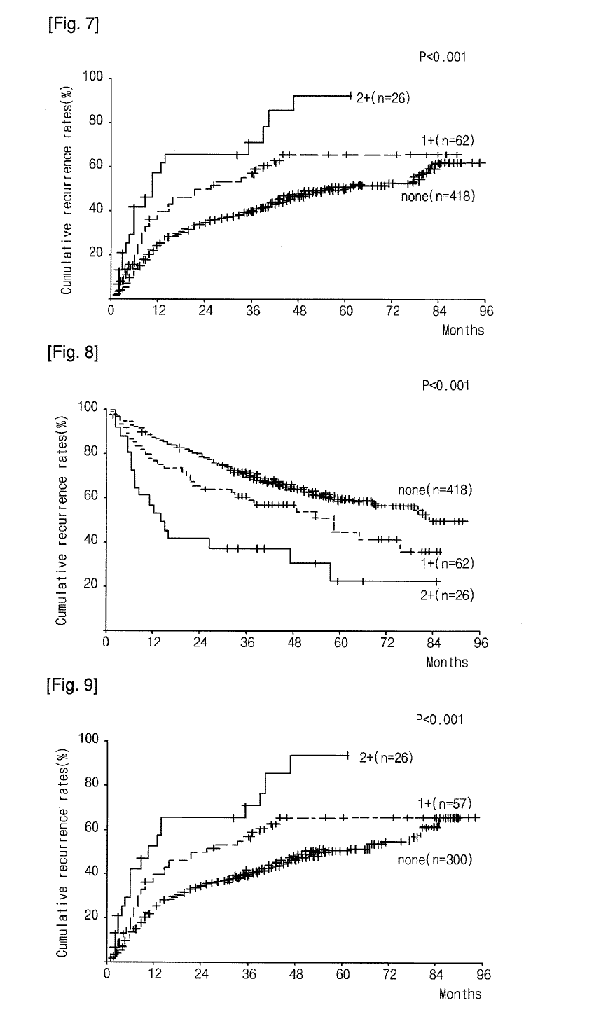

As illustrated in FIG. 7, the 1-year, 3-year, and 5-year cumulative recurrence rates of MTA1-positive HCC were much higher than those of MTA1-negative HCC. The cumulative recurrence rates in patients with high MTA1 expression levels (++) at 1, 3, and 5 years were 41%, 72%, and 93%, respectively, which were much higher than those in patients with MTA1 expression levels (+) (39%, 54%, and 65%, respectively) and negative MTA1 expression (25%, 39%, and 51%, respectively) levels.

As illustrated in FIG. 8, the 1-year, 3-year, and 5-year cumulative survival rates of patients with MTA1-positive HCC (71%, 54%, and 44%, respectively) were significantly shorter than those of patients with MTA1-negative HCC (89%, 72%, and 61%, respectively).

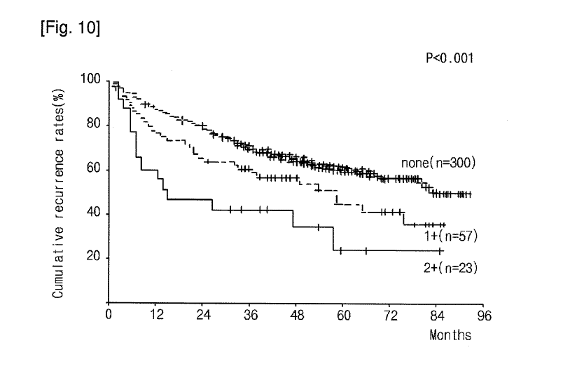

Also, as illustrated in FIGS. 9 and 10, for the patients with HBV infection, MTA1 expression levels were still significantly correlated with cumulative recurrence rates and cumulative survival rates.

MTA1, tumor size (>3.0 cm), histological differentiation (E-S III/IV), non-nodular tumor type, capsule invasion, portal vein thrombosis, and microvascular invasion were used for multivariate analysis of recurrence and survival.

As shown in Table 3, positive MTA1 staining (especially, ++), a larger tumor size (>3 cm in diameter), portal vein thrombosis, and microvascular invasion were independent prognostic factors for postoperative recurrence and survival.

TABLE 3

| | Recurrence | | Survival | |
|---|---|---|---|---|
| Variables | OR, 95% CI | P value | OR, 95% CI | P value |
| MTA1(+) | 1.510, 1.008-2.262 | 0.045 | 1.668, 1.092-2.549 | 0.018 |
| MTA1(++) | 3.248, 1.871-5.638 | <0.001 | 2.532, 1.383-4.634 | 0.003 |
| Tumor size (>3 cm) | 1.807, 1.230-2.655 | 0.003 | 2.336, 1.432-3.813 | 0.001 |
| Non-nodular type PVT | — | NS | — | NS |
| PVT | 2.220, 1.454-3.390 | <0.001 | 1.752, 1.110-2.763 | 0.016 |
| Capsular invasion | — | NS | 1.421, 0.970-2.082 | 0.072 |
| E-S grade (III/IV) | — | NS | — | NS |
| MVI | 1.719, 1.212-2.438 | 0.002 | 1.550, 1.061-2.266 | 0.023 |

9) Extrahepatic metastasis according to MTA1 expression levels

Of a total of 446 patients who undertook an evaluation about HCC at the last examination, 48% (213/446) had no recurrence. Single and multiple intrahepatic metastases were in 21% (95/446), and 17% (77/446), respectively. Extrahepatic metastasis occurred in 14% (61/446).

Interestingly, incidence of positive MTA1 staining (+ or ++) in each group was 12% (26/213), 19% (18/95), 23% (18/77), and 31% (19/61), respectively. Extrahepatic metastasis occurred more frequently in the MTA1-positive group (+ or ++) than in the negative group.

While the present inventive concept has been particularly shown and described with reference to exemplary embodiments thereof, it will be understood by those of ordinary skill in the art that various changes in form and details may be made therein without departing from the spirit and scope of the present inventive concept as defined by the following claims.

### SEQUENCE LISTING

A base sequence of MTA1 gene is set forth in SEQ ID NO: 1, and

An amino acid sequence of MTA1 protein is set forth in SEQ ID NO: 2.

1. A method of diagnosing the recurrence or possibility of recurrence of hepatocellular carcinoma after hepatocellular carcinoma surgery, the method comprising:
measuring expression levels of a metastatic tumor antigen 1 or metastasis associated 1(MTA1) protein in a biological sample taken from a patient in which hepatocellular carcinoma recurs or is likely to recur after hepatocellular carcinoma surgery; and
comparing the measured expression levels of the MTA1 protein with those of a normal control group.

2. The method of claim 1, wherein the expression levels of the sample are 25% higher than those of the normal control group.

3. A method of screening a drug for preventing the recurrence of hepatocellular carcinoma after hepatocellular carcinoma surgery and treating hepatocellular carcinoma, the method comprising:
contacting a metastatic tumor antigen 1 or metastasis associated 1(MTA1) protein with candidate drugs (step 1);
measuring expression levels of the MTA1 protein that have contacted the candidate drugs (step 2);
measuring expression levels of the MTA1 protein in the absence of the candidate drugs (step 3); and
comparing the expression levels of step 2 with the expression levels of step 3 to select a candidate drug that reduce expression levels of the MTA1 protein (step 4).
